(19) United States
(12) Patent Application Publication
(10) Pub. No.: US 2017/0281378 A1
(43) Pub. Date: Oct. 5, 2017

(54) STENT

(71) Applicant: TERUMO KABUSHIKI KAISHA, Tokyo (JP)

(72) Inventors: Yutaro SHINTAKU, Tokyo (JP); Takashi KUMAZAWA, Fujinomiya-city (JP); Yuusuke SEKINE, Chigasaki-city (JP)

(73) Assignee: TERUMO KABUSHIKI KAISHA, Tokyo (JP)

(21) Appl. No.: 15/625,332

(22) Filed: Jun. 16, 2017

Related U.S. Application Data
(63) Continuation of application No. PCT/JP2015/084319, filed on Dec. 7, 2015.

(30) Foreign Application Priority Data
Dec. 26, 2014 (JP) 2014-266471

(51) Int. Cl. A61F 2/915; A61F 2/958; A61F 2/04; A61F 2/954

(52) U.S. Cl. CPC A61F 2/915; A61F 2/954; A61F 2/958; A61F 2/04; A61F 2002/91583; A61F 2210/0004; A61F 2250/0071; A61F 2002/825

(57) ABSTRACT

A stent includes a strut formed into a cylindrical shape and extending in an axial direction. The strut includes outer peripheral portions extending around the axial and circumferential directions of the cylindrical shape. The outer peripheral portions are spaced apart from one another with gaps formed between adjacent outer peripheral portions. The strut includes a connection portion connecting the outer peripheral portions to each other in one of the gaps formed by the adjacent outer peripheral portions. The outer peripheral portions and the connection portion of the strut are integrally formed of a biodegradable polymer A portion of the strut includes a fragile portion which is snore fragile than other portions of the strut.

### CROSS-REFERENCES TO RELATED APPLICATIONS

This application is a continuation of International Application No. PCT/JP2015/084319 filed on Dec. 7, 2015, and claims priority to Japanese Patent Application No. 2014-266471 filed, on Dec. 26, 2014, the entire content of both of which is incorporated herein by reference.

### TECHNICAL FIELD

The present invention generally relates to a stent and a method of using a stent.

### BACKGROUND DISCUSSION

A stent is a medical device used to treat various diseases caused by a stenosed or occluded lumen of a blood vessel. A stent may he used for securing a cavity by widening a stenosed or occluded site. The stent has a substantially cylindrical shape whose outer periphery is formed in a mesh shape. The stent is configured to expand outward in a radial direction (i.e., radially outward). When the stent is positioned at a body lumen in a living body and expanded radially outward, an expansion holding force for securing the cavity is applied to the lumen.

The stent is used for not only a linear portion of a lumen, but also a bifurcated portion of the lumen (i.e., a branched or forked portion of the lumen). When the stent is used in the bifurcated portion of a lumen, for example, as disclosed in Japanese Patent Application Publication No. 2011-245001, a portion of the stent is broken, and an outer peripheral gap portion of the stent is broadened, thereby securing a route for communicating with the lumen of a bifurcated target.

### SUMMARY

When a stent formed of a biodegradable polymer is used for the bifurcated portion, if an, excessive force is forcibly applied when the outer peripheral gap portion of the stent is broadened, the overall stent may randomly collapse instead of only the portion of the stent intended to break. This collapse may occur because the biodegradable polymer is generally weaker in strength than metal. There is thus a possibility that the desired expansion holding force may not be applied to the lumen.

The stent disclosed in this application helps address this problem. The disclosed stent can prevent the overall stent from randomly collapsing when the stent is expanded even if the stent is formed of a biodegradable polymer.

A stent disclosed in this application has a configuration which is formed into a cylindrical shape by a strut. The strut has outer peripheral portions extending around an axial direction of the cylindrical shape and a connection portion connecting the outer peripheral portions to each other in a gap formed by the outer peripheral portions. The outer peripheral portions and the connection portion are integrally formed of a biodegradable polymer. In addition, a portion of the strut includes fragile portion which is more fragile than other different portions of the strut. The fragile portion includes a material that is the same as that of the other portions, and the biodegradable polymer itself is fragile.

Another stent disclosed in this application includes a stent body comprising a strut wound in a cylindrical shape. The stent body extends in an axial direction from a proximal end to a distal end. The strut is wound to form a plurality of outer peripheral portions that are spaced apart from one another with a gap being between adjacent outer peripheral portions. The stent includes a plurality of connection portions that each connects two of the adjacent outer peripheral portions to one another. The stent has a fragile portion located in one of the connection portions. The fragile portion is configured to break at a lower tensile force than any other portions of the strut. The strut, the connection portions and the fragile portion are a biodegradable polymer.

In another aspect, this application involves a method that includes inserting a stent and a first balloon catheter into a living body. The first balloon catheter includes a balloon, which has an outer surface. The balloon is deflated when the first balloon catheter is inserted into the luring body. The stent is directly on the outer surface of the balloon and possesses a contracted outer diameter. The stent is a biodegradable polymer. The stent includes outer peripheral portions with gaps between the outer peripheral portions and a fragile portion. The method further includes moving the first balloon catheter while the stent is directly on the outer surface of the balloon to a first branch of a stenosed site in a bifurcated body lumen in the living body and widening the first branch of the stenosed site of the bifurcated body lumen by inflating the balloon of the first balloon catheter so that the balloon expands radially outward. The stent on the outer surface of the balloon expands radially outward when the balloon is inflated so that the stent possesses an expanded outer diameter which is greater than the contracted outer diameter. The method includes deflating the balloon of the first balloon catheter. The stent maintains the expanded outer diameter in the first branch of the stenosed site in the bifurcated body lumen. The method includes inserting a second balloon catheter into the living body, moving the second balloon catheter into the interior of the stent and through one of the gaps between the outer peripheral portions of the stent while the stent is in the first branch of the stenosed site in the bifurcated body lumen so that the second balloon catheter enters a second branch of the stenosed site in the bifurcated body lumen and widening the second branch of the stenosed site in the bifurcated body lumen by inflating the balloon of the second balloon catheter so that the balloon of the second balloon catheter expands radially outward. The fragile portion of the stent breaks when the balloon of the second balloon catheter inflates.

According to the stent configured as described above, since the fragile portion is more easily broken, a strong force is less likely to be applied to other portions different from the fragile portion. It is thus possible to prevent the overall stent from randomly collapsing when expanded, even if the stent is formed of the biodegradable polymer.

### BRIEF DESCRIPTION OF THE DRAWINGS

FIG. 1 is a perspective view of a stent embodiment.

FIG. 2 is a development view in which a portion of an outer periphery of the stent shown in FIG. 1 is developed by being cut into a linear shape along an axial direction.

FIG. 3 is a partially enlarged view illustrating an enlarged fragile portion.

FIG. 4 is a stent development view illustrating an arrangement of the fragile portions in the overall stent, and a state where a portion having no fragile portion includes a helical shape.

FIG. 5 is a perspective view illustrating the stent shown in FIG. 1 mounted on a balloon catheter.

FIG. 6 is a perspective view illustrating the stent being expanded by a balloon.

FIG. 7 is a perspective view illustrating the balloon being deflated after he stent is expanded.

FIG. 8 is a perspective view illustrating the balloon being inserted into an outer peripheral gap of the expanded stent.

FIG. 9 is a partially enlarged view illustrating an enlarged outer periphery of the stent into which the balloon is inserted.

Set forth below with reference to the accompanying drawings is a detailed description of embodiments of a stent and a method of using a stent representing examples of the inventive stent and inventive method disclosed here. Dimensional proportions in the drawings are exaggerated and different from actual proportions for convenience of description.

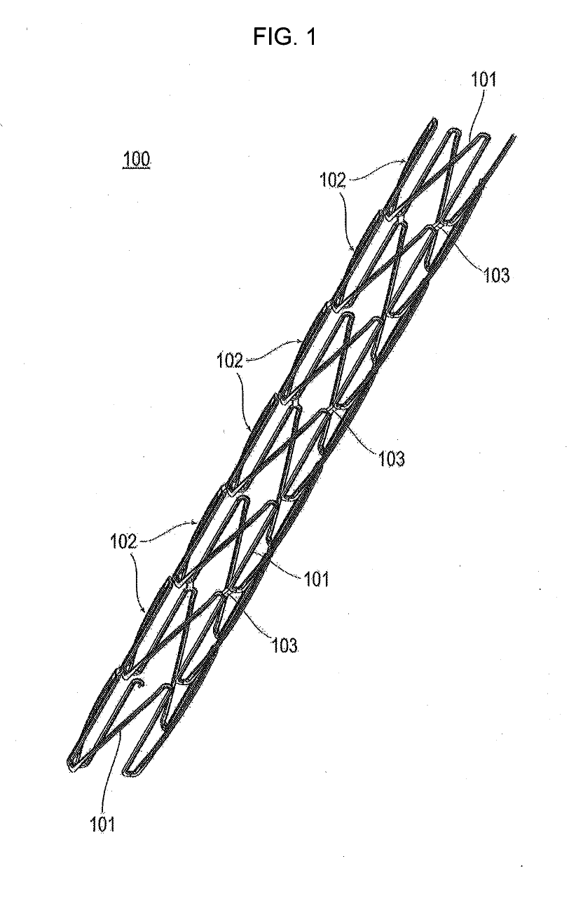

As illustrated in FIG. 1, one embodiment of the stent 100 has a strut 101 which is a linear configuration element. The stent 100 has a configuration in which a cylindrical shape is formed by the strut 101 (i.e., the stent 100 is cylindrically shaped). The strut 101 includes outer peripheral portions 102 extending along an axial direction of the cylindrical shape. The strut 101 also includes a connection portion 103 connecting the outer peripheral portions 102 to each other.

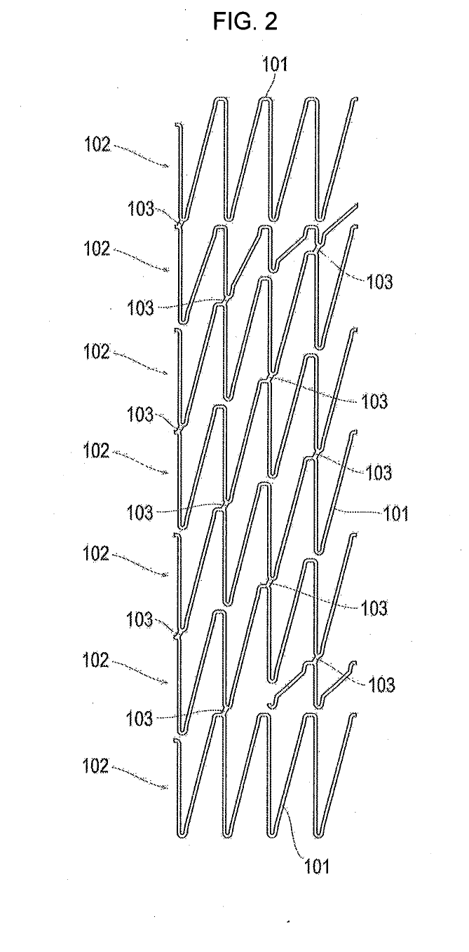

The connection portion 103 is disposed in a gap formed between the outer peripheral portions 102 as illustrated in FIG. 2.

For example, the outer peripheral portion 102 includes at least one of a helical portion extending in a helical shape around the axial direction (vertical direction in FIG. 2) of the stent 100, and endless annular portions extending around the axial direction of the stent 100. For example, in one embodiment of a stent 100, the annular portions are disposed at both ends in the axial direction of the stent 100 (i.e., the distal and proximal ends), and the helical portion is disposed between the annular portions at each of the ends. However, the arrangement of the annular portion(s) and/or the helical portion(s) is not particularly limited.

The connection portion 103 connects the outer peripheral portions 102 to each other so that the outer peripheral portions 102 are coaxially aligned in the axial direction of the stent 100. The arrangement of the connection portions 103, however, is not particularly limited. In addition, the number of connection portions 103 is not particularly limited.

The outer peripheral portion 102 and the connection portion 103 are integrally formed of a biodegradable polymer. The biodegradable polymer is a polymer which gradually biodegrades inside a living body. As long as the polymer does not adversely affect the inside of the living body (e.g., a human being or an animal), the biodegradable polymer is not particularly limited.

For example, the biodegradable polymer may preferably be at least one polymer selected from a group including aliphatic polyester, polyester, polyanhydrides, polyorthoester, polycarbonate, polyphosphazenes, polyphosphate ester, polyvinyl alcohol, polypeptides, polysaccharide, protein, and cellulose, a copolymer obtained by optionally copolymerizing a monomer configuring the above-described polymer, and a mixture of the polymers and/or the copolymers (here, the mixture means a broad concept including a compound such as a polymer alloy). Among these, it is preferable to use the aliphatic polyester since the aliphatic polyester is less reactive in a biological tissue and can control degradation inside the living body.

For example, the aliphatic polyester can include at least one polymer selected from a group including polylactic acid, polyglycolic acid, and polycaprolactone, a copolymer obtained by optionally copolymerizing the monomer configuring the above-described polymer, and the mixture of the polymers and/or the copolymers. The aliphatic polyester, however, is not limited to these polymers.

The weight-average molecular weight of the overall strut 101 (including he outer peripheral portion 102 and the connection portion 103) may be 10,000 or greater. However, the weight-average molecular weight of the overall strut 101 is not limited to any particular amount. The weight-average molecular weight of the overall strut 101 is preferably 10,000 to 1,000,000 more preferably 20,000 to 500,000, and even more preferably 50,000 to 200,000.

The method of measuring the weight-average molecular weight, for example, includes GPC, a light scattering method, a viscosity measurement'method, and TOF mass spectrometry (TOFMASS).

The stent 100 has a configuration in which the overall strut 101 is integrally formed of the biodegradable polymer. Accordingly, after fulfilling a stent 100 function such as restraining a rate of blood vessel occlusion and restenosis in an acute phase, the stent 100 vanishes (disintegrates) after being degraded and absorbed in the living body. Therefore, there is a low risk in restenosis and thrombotic complications in a last stage of the procedure.

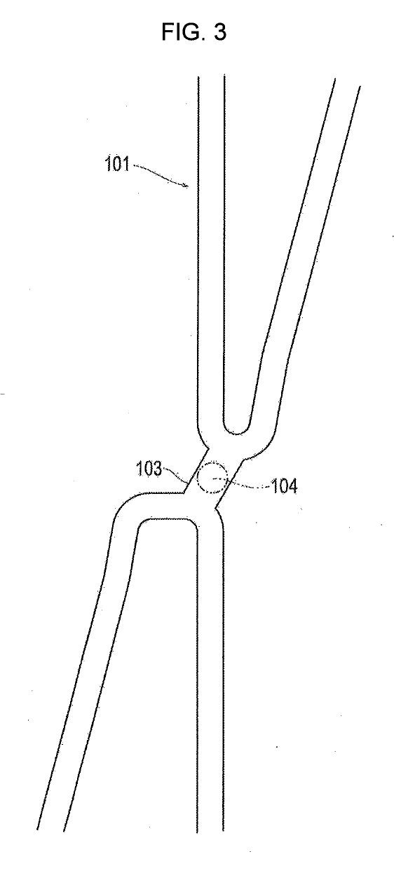

As illustrated in FIG. 3, a fragile portion 104 is formed in at least one connection portion 103. The fragile portion 104 is formed to be more fragile than other portions in the strut 101. In other words, the connection portion 103 having the fragile portion 104 is configured to break/rupture more quickly under lower tensile force than the other connection portions 103 that do not have the fragile portion 104. The fragile portion 104 may be formed in only a portion of the connection portion 103, or the overall (entire) connection portion 103 may be formed using the fragile portion 104. Any configuration may be adopted as long as the connection portion having the fragile portion is more likely to be broken than the other connection portion(s) that does not have the fragile portion.

For example, the fragile portion 104 is formed by causing a portion of the strut 101 of the biodegradable polymer to be fragile without forming the portion of the strut 101 by adding a material different from that of the biodegradable polymer forming the strut 101 as a separate member and without forming a notch or hole shape in a portion of the strut 101. As long as the fragile portion 104 is formed so that the biodegradable polymer itself is fragile while including the same material (biodegradable polymer) as that of other portions of the strut 101, a configuration is not particularly limited.

For example, the fragile portion 104 is formed so that the weight-average molecular weight of the fragile portion 104 is smaller than the eight-average molecular weight of the overall strut 101. The weight-average molecular weight of the fragile portion 104 is preferably 5% to 50% of the weight-average molecular weight of the overall strut 101.

The weight-average molecular weight of the fragile portion 104 may be reduced, for example, by applying the energy of an electron beam, a radiation beam, an infrared beam, or heat locally to a portion of he strut 101 (i.e., at the location that the fragile portion 104 is to be created).

The fragile portion 104 may additionally be formed by causing the biodegradable polymer itself to be fragile in such a way that a compounding ratio of the material of the biodegradable polymer in the fragile portion 104 is different than a compounding ratio of the material in other portions of the strut 101.

As an example, when the strut 101 is formed of a copolymer of polylactic and polyglycolic acid, the compounding ratio of polyglycolic acid in the fragile portion 104 can be increased compared to other portions.

The weight-average molecular weight of the fragile portion 104 or the compounding ratio of the material is different from other portions of the strut 101 as described above. The biodegradation rate of the fragile portion 104 can also be changed in addition to the strength of the fragile portion 104. For example, if the weight-average molecular weight of the fragile portion 104 is reduced the fragile portion 104 vanishes (disintegrates or degrades) more quickly than other portions of the strut 101.

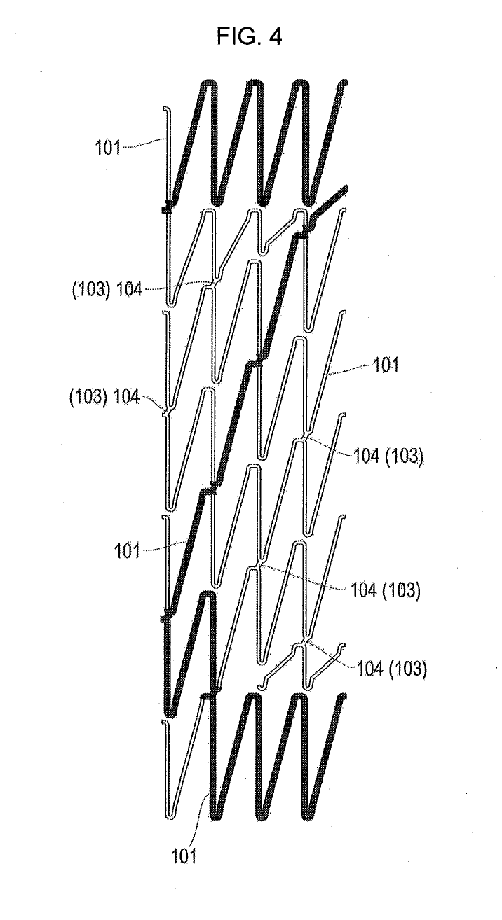

As illustrated in FIG. 4, the fragile portion 104 of an embodiment of the stent 100 is formed in a plurality of the connection portions 103. The strut 101 includes a helical shape (thick black line in FIG. 4) which ceaselessly continues from one end to the other end in the axial direction (vertical direction in FIG. 4) while avoiding the fragile portion 104. That is, the fragile portion 104 is not disposed in this continuous portion of the strut 101 that possesses the helical shape. The fragile portion 104 is not formed in the portion having the helical shape illustrated by the thick black line in FIG. 4. Accordingly, this portion is less likely to be broken.

Next, a method form of using the stent 100 is described.

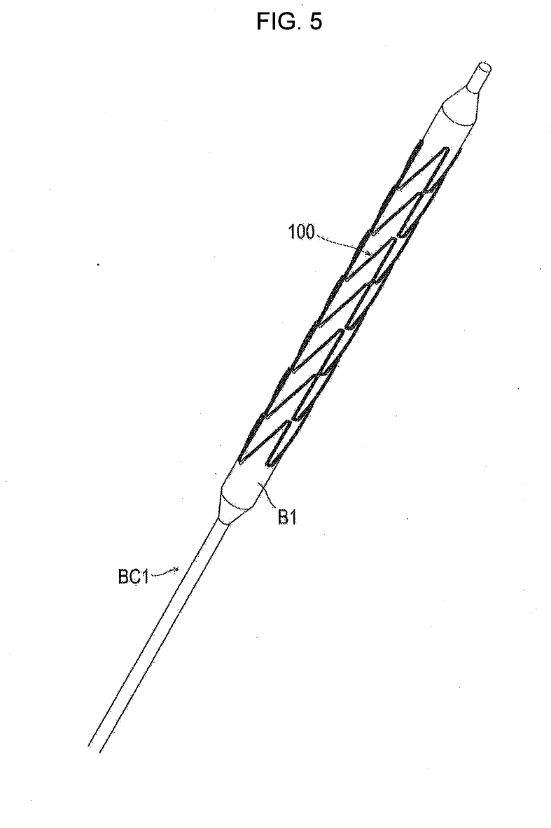

The stent 100 may be mounted on a balloon B1 of a balloon catheter BC1 as illustrated in FIG. 5 and delivered to a bifurcated portion of a body lumen (such as a blood vessel). When the stent 100 is being moved within the body to be delivered to the bifurcated portion of the body lumen, the balloon B1 is deflated and the stent 100 is contracted as shown, in FIG. 5. A known balloon catheter BC1 in the related art may be utilized. The balloon catheter BC1 and the stent 100 can also, be delivered to a desired position inside the body lumen, by using a known manual skill in the related art. Accordingly, detailed description of balloon catheters and catheter delivery techniques will be omitted,

The lumen that the tent 100 is used in/delivered to is not limited to a blood vessel. For example, the lumen may be a biliary duct, a bronchial tube, an esophagus, other gastrointestinal tracts, and a urethra.

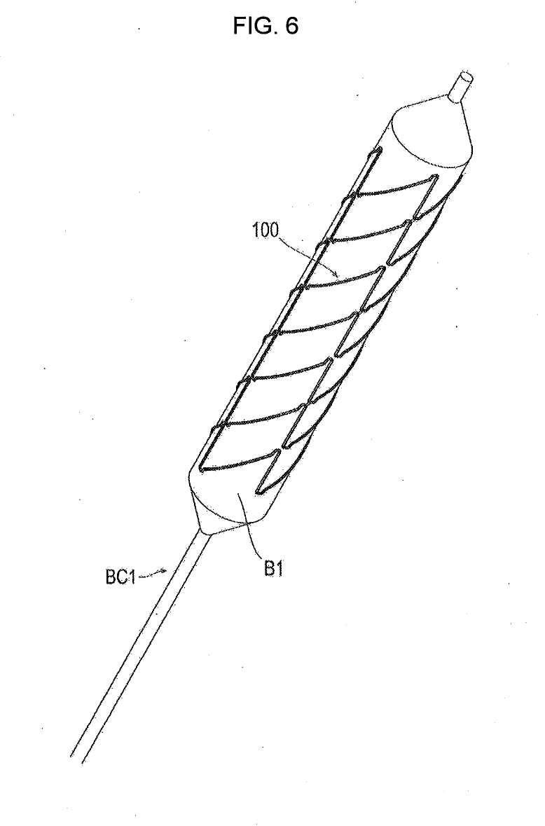

After the stent 100 is delivered to the bifurcated portion of the lumen, the balloon B1 is dilated (inflated) as illustrated in FIG. 6. The balloon B1 expands radially outwardly and so the stent 100 on the outer surface of the balloon B1 also expands radially outwardly. The balloon B1 and the stent 100 may thus widen a stenosed site or an occluded site appearing in the bifurcated portion.

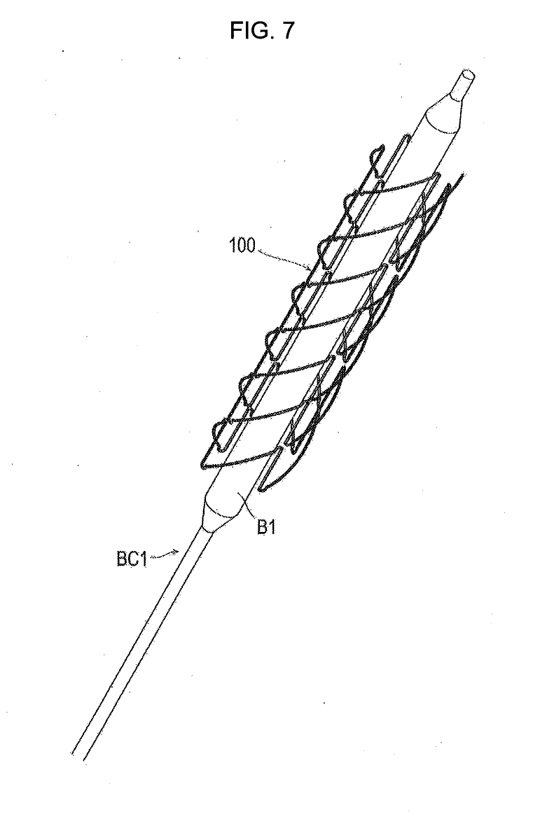

After the stent 100 is expanded, the balloon 61 is deflated as illustrated in FIG. 7. When the balloon B1 deflated, the stent 100 maintains an expanded state. The expanded stent 100 secures a cavity in a living body by applying an expansion holding force to the body lumen.

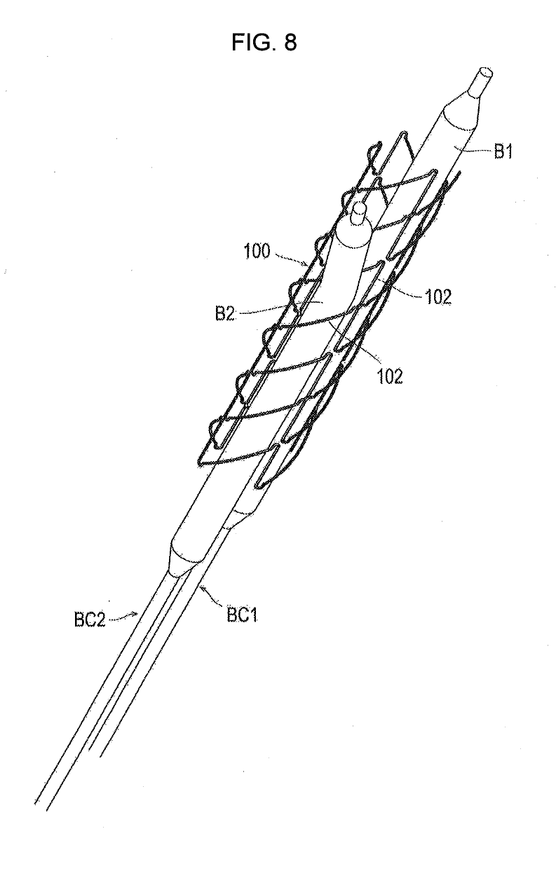

A balloon B2 of another balloon catheter BC2 is then inserted into a gap formed by the outer peripheral portion 102 of the stent 100 as illustrated in FIG. 8. The balloon B2 is oriented in the direction of the other body lumen bifurcated from the body lumen into which the balloon B1 is inserted. In other words, the balloon B1 is in one portion of the bifurcated body lumen and the balloon B2 is in the other portion of the bifurcated body lumen. It is possible to use a balloon catheter known in the related art as the balloon catheter BC2.

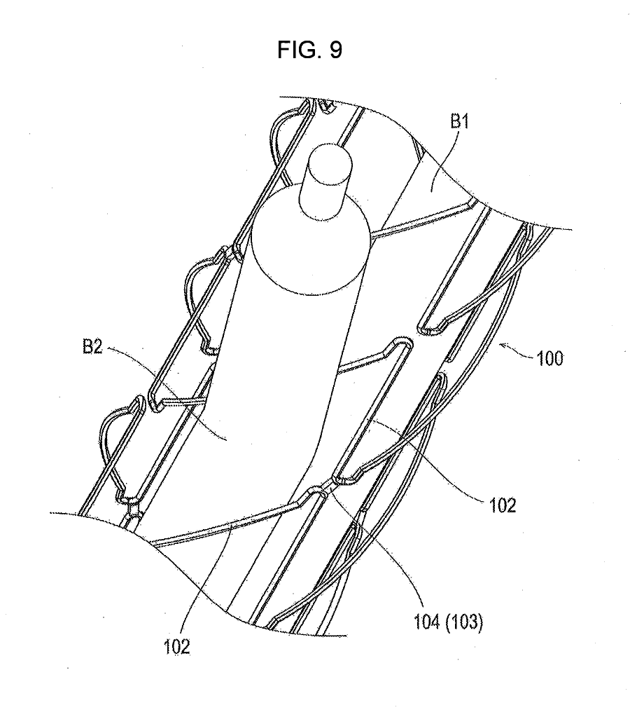

The balloon B2 dilates and applies a tensile force to the outer peripheral portion 102 and the fragile portion 104 which are located around the balloon B2 as illustrated in FIG. 9. When this tensile force is applied to the outer peripheral portion 102 and the fragile portion 104, the fragile portion 104 breaks. As a result, the gap formed by the outer peripheral portion 102 is broadened, thereby forming a large opening portion (i.e., larger than the other openings between adjacent sections of the strut) in the stent 100). A medical device such as the balloon catheter BC2 is thus able to be delivered to the other portion of the bifurcated body lumen serving as a bifurcated target.

The balloon B2 is dilated, and the balloon B1 is also dilated, thereby holding the stent 100 and pressing the stent 100 against an inner wall of the body lumen.

Next, an operation effect according to the present embodiment is described.

Since the fragile portion 104 of the stent 100 is broken (i.e., is configured to break before any other portions of the stent 100), a strong force is less likely to be applied to the other portions of the stent 100. For example, the outer peripheral portion 102 and the connection portion 103 having no fragile portion 104 do not receive a relatively strong force because the fragile portion 104 is broken. Therefore, it is possible to prevent the overall stent 100 from randomly collapsing (i.e., breaking at portions of the stent 100 other than the fragile portions 104) when expanded, even if the stent 100 is integrally formed of the biodegradable polymer.

The fragile portion 104 is formed in the embodiment illustrated in FIG. 4 in the connection portion 103 instead of the outer peripheral portion 102. Accordingly, the fragile portion 104 is restrained from vanishing due to the broken or degraded outer peripheral portion 102. Therefore, the stent 100 can effectively apply the expansion holding force to the lumen from the outer peripheral portion 102.

As illustrated by the thick black line in FIG. 4, the strut 101 includes a helical shape which continues from one end to another end in the axial direction (vertical direction in FIG. 4) while avoiding the fragile portion 104 (i.e., the continuous helical shape of the strut 101 from the distal end to the proximal end does not include or is devoid of any fragile portions 104). Therefore, even if the fragile portions 104 vanish (disintegrate) by being broken or preferentially degraded at a plurality of locations, the strut 101 remains connected from one end to another end in the axial direction. The stent 100 is thus prevented from being broken, thereby enabling the stent 100 to stably fulfill its function.

When the weight-average molecular weight of the fragile portion 104 is equal to or smaller than 50% of the eight-average molecular weight of the overall strut 101, the strength of the fragile portion 104 is further reduced compared to the, other portions of the strut 101. Accordingly, the fragile portion 104 can be easily broken (i.e., the fragile portion 104 is configured to break before the other portions of the strut 101).

When the weight-average molecular weight of the overall strut 101 is 10,000 or greater, the strength is ensured for the overall stent 100. The stent 100 can thus stably hold the widened lumen.

The compounding ratio of the material of the biodegradable polymer is set so that the fragile portion 104 has a different compounding ratio than the other portions of the strut 101. In this manner, when the strength of the fragile portion 104 is weakened, the fragile portion 104 can be easily broken.

Without being limited to the above-described embodiment, the present invention can be modified in various ways within the scope of the appended claims.

For example, the location for forming the fragile portion may be in portion of the strut which is different from the connection portion. For example, the fragile portion may be formed in the outer peripheral portion.

The location for using the stent described above is not limited to the bifurcated portion of the body lumen. The stent may be used in a location having no bifurcation in the lumen, for example, such as a linear site.

The detailed description above describes a stent and a method of using a stent. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a strut formed into a cylindrical shape and extending in an axial direction and possessing a circumferential direction;
the strut comprising outer peripheral portions extending around the axial and circumferential directions of the cylindrical shape, the outer peripheral portions being spaced apart from one another with gaps being formed between adjacent outer peripheral portions;
the strut comprising a connection portion connecting the outer peripheral portions to each other in one of the gaps formed by the adjacent outer peripheral portions;
the outer peripheral portions and the connection portion of the strut being integrally formed of a biodegradable polymer; and
a portion of the strut comprising a fragile portion which is ore fragile than other portions of the strut, the fragile portion including the biodegradable polymer that is the same as the biodegradable of the other portions of the strut, and the biodegradable polymer of the fragile portion itself is fragile.

2. The stent according to claim 1, wherein the fragile portion is formed in the connection portion.

3. The stent according to claim 1, wherein the strut includes a helical shape which ceaselessly continues from one end to the other end in the axial direction of the cylindrical shape while avoiding the fragile portion.

4. The stent according to claim 1, wherein a weight-average molecular weight of the fragile portion is equal to or smaller than 50% of a weight-average molecular weight of the overall strut.

5. The stent according to claim 4, wherein the weight-average molecular weight of the overall strut is 10,000 or greater.

6. The stent according to claim 1, wherein a compounding ratio of the biodegradable polymer in the fragile portion is different from a compounding ratio of the other portions of the strut.

7. A stent comprising:
a stent body comprising a strut wound in a cylindrical shape, the stent body extending in an axial direction from a proximal end to a distal end, the stent body possessing a radial direction and a circumferential direction;
the strut being wound to form a plurality of outer peripheral portions, the outer peripheral portions being spaced apart from one another with a gap being between adjacent outer peripheral portions;
a plurality of connection portions that each connect two of the adjacent outer peripheral portions to one another;
a fragile portion located in one of the connection portions, the fragile portion being configured to break at a lower tensile force than any other portions of the strut; and
the strut, the connection portions and the fragile portion being a biodegradable polymer.

8. The stent according to claim 7, wherein when the fragile portion breaks, the gap between the adjacent outer peripheral portions previously connected by the one of the connection portions increases.

9. The stent according to claim 7, wherein
the strut possesses a weight-average molecular weight,
the fragile portion possesses a weight-average molecular weight, and
the weight-average molecular weight of the fragile portion lower than the weight-average molecular weight of the strut.

10. The stent according to claim 9, wherein the weight-average molecular weight fragile portion is equal to or smaller than 50% of the weight-average molecular weight of the strut.

11. The stent according to claim 10, wherein the weight-average molecular weight of the strut is 10,000 or greater.

12. The stent according to claim 7, wherein a compounding ratio of the biodegradable polymer in the fragile portion is different from a compounding ratio of the biodegradable polymer in the any other portions of the strut.

13. The stent according to claim 7, further comprising:
a plurality of fragile portions at some of the connection portions.

14. The stent according to claim 13, wherein
the strut wound in the cylindrical shape comprises a helically-shaped segment, the helically-shaped segment extending continuously from the proximal end to the distal end of the stent body, and
the helically-shaped segment being devoid of the fragile portions.

15. The stent according to claim 7, wherein the strut comprises a first annular portion, a second annular portion and a helical portion, the first annular portion being at the proximal end of the stent body, the second annular portion being at the distal end of the stent body, and the helical portion extending from the second annular portion to the first annular portion in the axial direction.

16. The stent according to claim 7, wherein the biodegradable polymer includes at least one polymer selected from a group including aliphatic polyester, polyester, polyanhydrides, polyorthoester, polycarbonate, polyphosphazenes, polyphosphate ester, polyvinyl alcohol, polypeptides, polysaccharide, protein, and cellulose,

17. A method comprising:
inserting a stent and a first balloon catheter into a living body, the first balloon catheter comprising a balloon which has, an outer surface, the first balloon catheter extending in an axial direction and possessing a radial direction, the balloon being deflated when the first balloon catheter is inserted into the living body, the stent being directly on the outer surface of the balloon and possessing a contracted outer diameter, the stent being a biodegradable polymer, the stent comprising outer peripheral portions with gaps between the outer peripheral portions and a fragile portion;
moving the first balloon catheter while the stent is directly on the outer surface of the balloon to a first branch of a stenosed site in a bifurcated body lumen in the living body;
widening the first branch of the stenosed site of the bifurcated body lumen by inflating the balloon of the first balloon catheter so that the balloon expands radially outward, the stent on the outer surface of the balloon expanding radially outward when the balloon is inflated so that the stent possesses an expanded outer diameter which is greater than the contracted outer diameter;
deflating the balloon of the first balloon catheter, the stent maintaining the expanded outer diameter in the first branch of the stenosed site in the bifurcated body lumen, the stent possessing an interior;
inserting a second balloon catheter into the living body, the second balloon catheter comprising a balloon:
moving the second balloon catheter into the interior of the stent and through one of the gaps between the outer peripheral portions of the stent while the stent is in the first branch of the stenosed site in the bifurcated body lumen so that the second balloon catheter enters a second branch of the stenosed site in the bifurcated body lumen; and
widening the second branch of the stenosed site in the bifurcated body lumen by inflating the balloon of the second balloon catheter so that the balloon of the second balloon catheter expands radially outward, the fragile portion of the stent breaking when the balloon of the second balloon catheter inflates.

18. The method according to claim 17, wherein the first balloon catheter remains in the first branch of the stenosed site in the bifurcated body lumen while the second balloon catheter is inserted into the living body and inflated at the second branch of the stenosed site in the bifurcated body lumen.

19. The method according claim 17, wherein the stent comprises a strut wound in a cylindrical shape.

20. The method according to claim 19, wherein
the strut possesses a weight-average molecular weight,
the fragile portion possesses a weight-average molecular weight, and
the weight-average molecular weight of the fragile portion is lower than the weight-average molecular weight of the strut.
